(19) United States
(12) Patent Application Publication
(10) Pub. No.: US 2008/0234550 A1
(43) Pub. Date: Sep. 25, 2008

(54) Minimally Traumatic Portal

(76) Inventors: David T. Hawkes, Pleasant Grove, UT (US); Michael D. Ensign, Salt Lake City, UT (US); Thomas M. Sweeney, Sarasota, FL (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 12/051,551

(22) Filed: Mar. 19, 2008

Related U.S. Application Data
(63) Continuation-in-part of application No. 11/384,139, filed on Mar. 17, 2006.
(60) Provisional application No. 60/685,185, filed on May 26, 2005.
(60) Provisional application No. 60/703,606, filed on Jul. 29, 2005.
(60) Provisional application No. 60/918,859, filed on Mar. 19, 2007.
(60) Provisional application No. 60/982,013, filed on Oct. 23, 2007.

(51) Int. Cl. A61B 1/32

(52) U.S. Cl. 600/204; 606/108

(57) ABSTRACT

A less invasive access port for use in minimally invasive surgery allows for manipulation of the viewing angle into the working site in a transverse plane. According to one exemplary embodiment, the less invasive access port is designed to minimize the need for muscle retraction. Additionally, the less invasive access portal provides sufficient light, irrigation, suction and space for sundry medical instruments. According to one exemplary embodiment, a less invasive access port device includes a retractor assembly having four retractor blades secured in various positions by pins placed within slots on the retractor blades. A cannula includes integrated interfaces for light, irrigation and suction. A housing forms a collar around a top of the cannula and houses the light, irrigation and suction mechanisms. Instruments and implants may be passed through the cannula and into the working space created by the retractor assembly. Visualization of the working site can be attained under direct vision.

### RELATED APPLICATIONS

The present application is a Continuation-In-Part application of U.S. patent application Ser. No. 11/384,139, which application claims the benefit under 35 U.S.C. § 119(e) of U.S. Provisional Patent Application Nos. 60/685,185 filed on May 26, 2005 and 60/703,606 filed on Jul. 29, 2005. Furthermore, the present application claims the benefit under 35 U.S.C. § 119(e) of U.S. Provisional Patent Application No. 60/918,859 filed Mar. 19, 2007 which is titled “Minimally traumatic portal” and U.S. Provisional Patent Application No. 60/982,013 filed Oct. 23, 2007 titled “Ratcheting Retractor Blades & Flexible Tube Retention Sleeve for Access Port.” The above-mentioned patent applications are incorporated herein by reference in their entireties.

### FIELD

The present system and method relate to devices and methods for performing percutaneous surgeries, and more particularly, to a less invasive access portal for use in orthopedic spinal surgery.

### BACKGROUND

Traditionally, the surgical exposure employed to perform spinal surgery inflicts significant and long lasting damage to the surrounding soft tissues. Surgical exposure, commonly referred to as an ‘open’ procedure, relies on retraction of muscles to open a channel to the underlying bony structures. Surgical retractors are often used to provide the operating channel. Common surgical retractors used in the art today include rakes, forks, and hooks of varying sizes and shapes. Normally, the hooks are constructed of a stainless steel or latex-free silicon so that they may be used in the sterile environment of the surgery. While such retractors as rakes or hooks are useful for certain types of injury, extreme care must be used to ensure that the retractor does not cause additional damage to the wound. In addition, use of the surgical retractor may require two, three, or more additional assistants to the physician, with appropriate training, in order to hold the retractor in the correct position so that the site of the surgery is more easily accessible to the physician. Other traditional surgical retractors are inserted into the surgical site and then one or more arms are spread in order to open the insertion site for further access by the physician. These traditional retractors are generally bulky, require substantial training and skill to operate, and user error may increase the difficulty and the time for the surgery. Traditional retraction using the above-mentioned retractors is recognized to cut-off circulation to the muscles and often results in post-operative pain and long-term degradation of muscle function.

Recently, minimally invasive techniques have been developed to reduce the intra-operative damage and reduce the post-operative recovery time. In minimally invasive surgery (MIS), a desired site is accessed through portals rather than through a significant incision. Various types of access portals have been developed for use in MIS. Many of the existing MIS access portals, such as those described in U.S. Pat. Nos. 4,573,488 and 5,395,317 issued to Kambin, can only be used for a specific procedure. Other prior art portals, such as those described in U.S. Pat. No. 5,439,464 issued to Shapiro, call for the placement of multiple portals into the patient, adding complexity to the portal placement as well as obstructing the operating space.

### SUMMARY

According to one exemplary embodiment of the present system and method, a less invasive access port includes a retractor having a plurality of members; each member being coupled to adjacent members. When the retractor members are positioned for insertion into the tissue, the distal portions are adjacent to each other. The retractor is then inserted into the tissue, adjacent the site for a desired medical procedure. Pins inserted in slots on each member are configured to secure the distal ends of the retractor members adjacent to each other. Upon insertion of the retractor into the desired location, the pins are allowed to slide up a channel formed in each of the retractor members, which expands the distal portion to create a working space inside the tissue

In one exemplary embodiment, the less invasive access port is configured for use in minimally invasive surgery and allows for manipulation of the viewing angle into the working site in any desired angle including both an axial plane and a mediolateral plane. Further, the present exemplary less invasive access port is configured to minimize muscle retraction. According to further aspects of the exemplary less invasive access port, sufficient light, irrigation, suction, and space for sundry medical instruments is provided through the access port.

In one exemplary embodiment, the channel formed in each retractor member is configured with teeth, allowing the pins to be ratcheted to a desired location. This enables the retractor members to be positioned and maintained in a partially expanded state.

Further, a housing having a port there through is configured to engage the retractor, providing integrated light, irrigation, and suction mechanisms. Once engaged with the retractor, the housing is free to pivot flexibly within the two-piece retractor, thus providing access to the entire working site through the port. According to aspects of this embodiment, instruments and implants may be passed through the port and into the working space created by the retractor. According to aspects of one exemplary embodiment, visualization of the working site is preferably attained under direct vision.

Moreover, according to one exemplary embodiment, the present exemplary less invasive access port provides for a method of performing spinal surgery that includes percutaneously inserting one or more screws in a bony portion of a spine, placing a trocar onto the bony portion of the spine to provide access to the working site, inserting a retractor over the trocar down to the working site, inserting a cannula into the retractor, and expanding the retractor to expose the working site. According to one exemplary embodiment, the insertion of the one or more screws, as well as insertion of the trocar, retractor, and the cannula are performed in the plane lateral to the multifidus in the fascial plane.

### BRIEF DESCRIPTION OF THE DRAWINGS

The accompanying drawings illustrate various exemplary embodiments of the present system and method and are a part of the specification. Together with the following description, the drawings demonstrate and explain the principles of the present system and method. The illustrated embodiments are examples of the present system and method and do not limit the scope thereof.

FIG. 1 is a drawing of a less invasive access port with retractor members expanded, according to one exemplary embodiment.

FIG. 2 is a drawing of a less invasive access port with retractor members contracted, according to one exemplary embodiment.

FIG. 3 is a trocar used with the less invasive access port, according to one exemplary embodiment.

FIG. 4 is a partial cut-away side view of a retractor inserted into a patient, according to one exemplary embodiment.

FIGS. 5A and 5B are drawings of a retractor assembly with retractor members contracted and with retractor members expanded, respectively, according to one exemplary embodiment.

FIG. 6 is a drawing showing a retractor assembly having teeth within the slots allowing a pin to be ratcheted to a desired location, according to one exemplary embodiment.

FIG. 7 is an isometric view of a cannula assembly, according to one exemplary embodiment.

FIG. 8 is an isometric view of a cannula assembly having a leyla arm attachment thereon, according to one exemplary embodiment.

FIG. 9A is a bottom isometric view of the cannula assembly of FIG. 7, according to one exemplary embodiment.

FIG. 9B is an isometric view of the cannula sleeve of FIG. 7, according to one exemplary embodiment

FIG. 10 is an isometric view of a cannula assembly introduced over a trocar to engage a retractor assembly, according to one exemplary embodiment.

FIG. 11 is an isometric view of the less invasive access port in a deployed position (retractor members expanded) prior to removal of the trocar, according to one exemplary embodiment.

FIG. 12 is a flow chart illustrating a method for performing spinal surgery using the present less invasive access port, according to one exemplary embodiment.

FIG. 13 is a top view illustrating the insertion of a pedicle screw in the fascial plane lateral to the multifidus, according to one exemplary embodiment.

FIGS. 14A-14D are side elevational views of a retractor in various deployed, undeployed and positions there between during a spinal surgery procedure, according to exemplary embodiments.

FIGS. 15A and 15B are side elevational views of the cannula assembly, according to exemplary embodiments.

FIGS. 16A-16D are side elevational views of a cannula assembly at an angle relative to the retractor assembly through the use of a flexible sleeve (FIGS. 15A and 15C) or a flexible connection (FIGS. 15B and 15D).

FIGS. 17A-17C are exploded, side retracted, and side expanded views of a less invasive access port, according to one exemplary embodiment.

FIG. 18A is a perspective view of a ratchet latch, according to one exemplary embodiment.

FIGS. 18B and 18C are side cross-sectional views of a retractor assembly, according to one exemplary embodiment.

Throughout the drawings, identical reference numbers designate similar but not necessarily identical elements.

### DETAILED DESCRIPTION

The present specification describes a system and a method for performing spinal surgery using minimal invasive surgery (MIS) techniques. Further, according to one exemplary embodiment, the present specification describes a less invasive access port that allows for mediolateral pivot of a cannula member while maintaining a retractor locking mechanism outside the wound. Additionally, the exemplary less invasive access port device described herein provides integrated light, suction, and irrigation capabilities, without interfering with the operational access port. The functionality of the less invasive access port described herein allows for a surgical method wherein any number of pedicle screws are inserted prior to the insertion of the less invasive access port. Moreover, the present exemplary MIS technique includes insertion of the pedicle screw(s) and the less invasive access port in the fascial plane lateral to the multifidus, thereby greatly reducing damage to soft tissue during surgery. Further details of the present exemplary system and method will be provided below.

By way of example, pedicle screw systems may be fixed in the spine in a posterior lumbar fusion process via minimally invasive surgery (MIS) techniques. The systems are inserted into the pedicles of the spine and then interconnected with rods to manipulate (e.g., correct the curvature, compress or expand, and/or structurally reinforce) at least portions of the spine. Using the MIS approach to spinal fixation and/or correction surgery has been shown to decrease a patient's recovery time and reduce the risks of follow-up surgeries.

The ability to efficiently perform spinal fixation and/or correction surgeries using MIS techniques is enhanced by the use of the less invasive access port and its associated surgery method provided in accordance with the present exemplary systems and methods, which systems and methods provide a number of advantages over conventional systems, as will be detailed below.

In the following description, for purposes of explanation, numerous specific details are set forth in order to provide a thorough understanding of the present system and method for a less invasive access port system. It will be apparent, however, to one skilled in the art that the present method may be practiced without these specific details. In other instances, well-known structures associated with the less invasive access port have not been shown or described in detail to avoid unnecessarily obscuring descriptions of the embodiments. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

### Exemplary Overall Structure

While the present system and method may be practiced by or incorporated into any number of systems, the present system and method will be described herein, for ease of explanation only, in the context of a less invasive access portal for use in orthopedic spinal surgery; providing a channel to the underlying bony structures of the spine while minimizing trauma to the overlying tissues. According to aspects of the present exemplary system and method, the less invasive access portal is able to minimize the need for muscle retraction. Additionally, according to one exemplary embodiment, the less invasive access portal provides sufficient light, irrigation, suction and space for sundry medical instruments. The features and advantages of the exemplary systems and methods will be set forth in the description which follows, and in part will be apparent from the description.

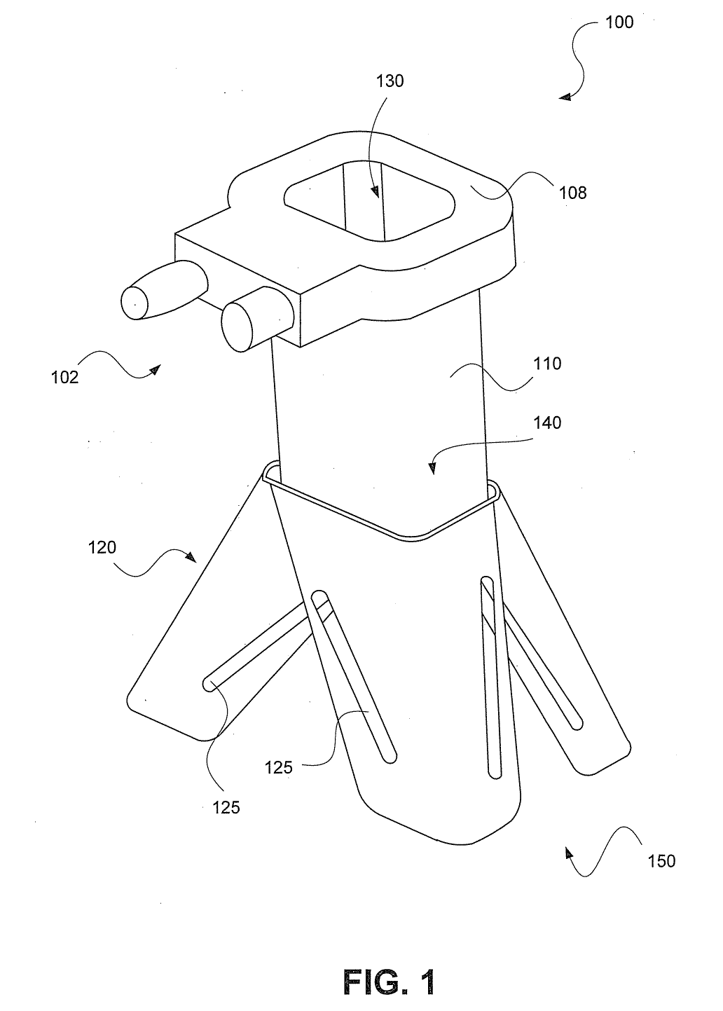

FIG. 1 shows an assembled less invasive access port device (100) in a deployed position, according to one exemplary embodiment. As shown, the exemplary less invasive access port device (100) includes a retractor assembly (120) having a proximal (140) and a distal end (150). Additionally, a cannula sleeve (110) is coupled to the proximal end (140) of the retractor assembly (120). An inner wall of the cannula sleeve (110) defines an access port (130). According to one exemplary embodiment, instruments and implants may be passed through the access port (130) defined by the cannula sleeve (110) and into a working space created by the retractor assembly (120). Further, as illustrated in FIG. 1, the cannula sleeve (110) of the less invasive access port device (100) includes integrated interfaces (102) for light, irrigation and suction. According to the exemplary embodiment shown in FIG. 1, a housing (108) forms a collar around a top of the cannula sleeve (110) and houses the light, irrigation and suction interface (102), as well as the light, irrigation, and suction channels. The cannula sleeve (110) and the housing (108) collar together are referred to herein as the cannula assembly. According to one exemplary embodiment described in further detail below, the cannula sleeve (110) is flexibly coupled to the retractor assembly (120) such that the cannula sleeve may be rotated to a desired angle relative to the retractor assembly once a trocar and k-wire are removed. The ability for the cannula sleeve (110) to be flexibly positioned at an angle relative to the retractor assembly (120) provides access to the entire working site defined by the retractor assembly (120). According to one exemplary embodiment, visualization of the working site is attained under direct vision. Further details of each component of the less invasive access port device (100), their assembly, and the tools used in conjunction therewith will be provided below with reference to FIGS. 1-16.

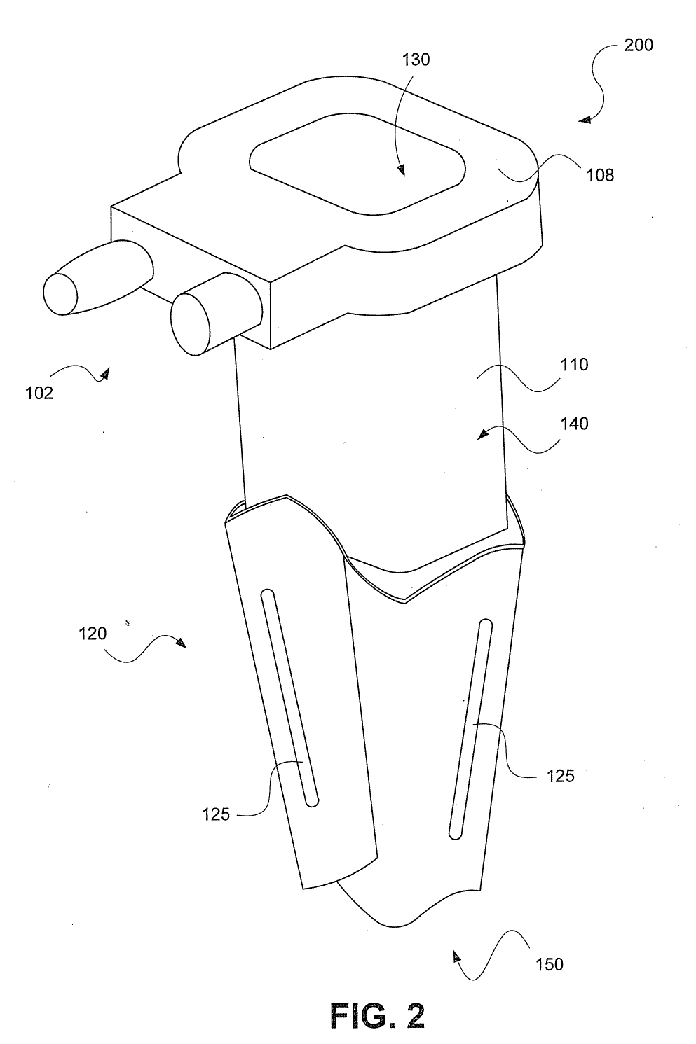

FIG. 1 illustrates a fully assembled less invasive access device in a deployed position. As shown in FIG. 1 a deployed position is defined as a position in which the retractor members of the retractor assembly (120) are extended at least partially. As shown in FIG. 2, the retractor members are closed or fully contracted. As illustrated in FIG. 1, the retractor assembly (120) consists of four retractor members; however, it is within the scope of this disclosure to utilize any number of retractor members for a specific application. FIGS. 1 and 2 also show a slot (125) extending from the distal end (150) of the retractor assembly to the proximal end (140). The slot or slit (125) does not extend to either extreme (proximal or distal end) and may also include notches (not shown).

The slots (125) are used in conjunction with a pin (not shown); the pin is inserted in the slot through two slots, one slot each from two different retractor members. This is best seen in FIG. 2 where the retractor members are in a contracted state in can be seen that the slots (125) are aligned; a pin positioned near the bottom of the slot will secure the retractor members from expanding into a deployed state. The slot may have grooves or notches capable of securing the pin from inadvertently sliding. Sliding the pin up the slot (125) toward the proximal end (140) of the retractor assembly (120) causes the retractor members to expand into a deployed state.

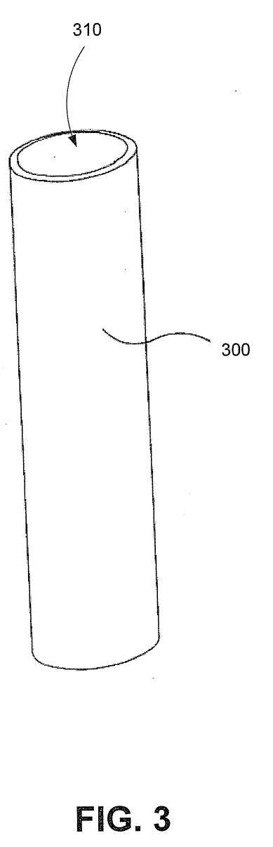

As mentioned above, the exemplary less invasive access port device (100) may be slideably positioned into a work area by the use of a trocar. FIG. 3 illustrates an exemplary trocar (300) for use with the less invasive access port device (100) of FIG. 1. In operation, a k-wire may be initially inserted into the soft tissues. Any number of pedicle screws may then be percutaneously inserted into a desired bone mass. The trocar (300) may then be placed over the k-wire to dilate the soft tissues and provide access to a desired working site. As used herein, the trocar (300) may be any number of stylets used for exploring or dilating tissue. According to one exemplary embodiment, the trocar (300) includes a triangular point on one end. However, the point of the trocar (300) used in connection with the present exemplary less invasive access port device (100) may assume any number of geometric profiles.

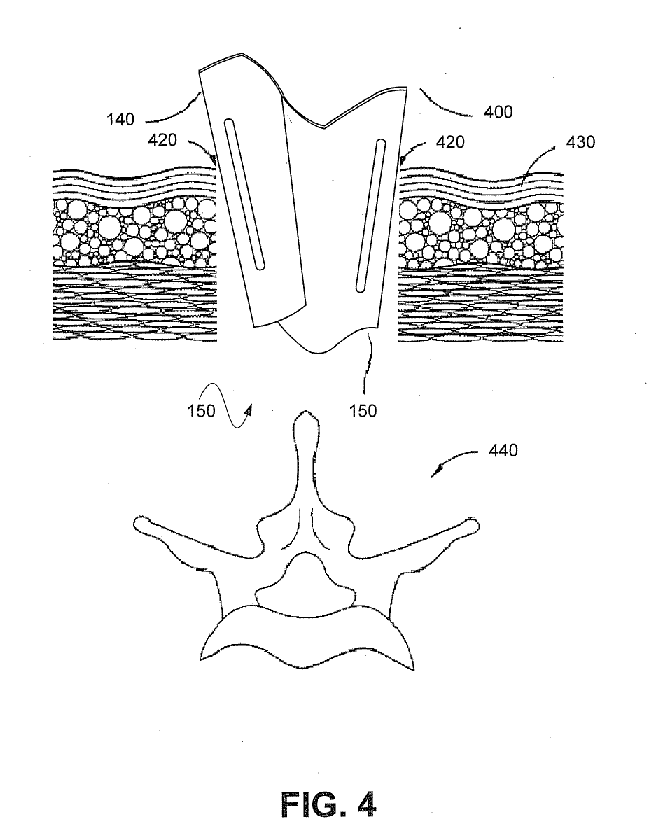

After placing a trocar in the desired working site, the retractor assembly is placed within the area, as shown in FIG. 4. When inserted into an opening (420) in the skin (430), the distal end (150) is inserted first with the retractor assembly (120) retracted; the pin is in the lowest portion of the slot (125) and the retractor members are fully closed, to be minimally invasive. Once inserted, the retractor assembly (120) may be actuated to provide workable access to a vertebra (440) or other desired structure. By sliding the pin up, towards the proximal end, the retractor members are released allowing them to expand, see FIG. 1.

As the retractor members spread apart from each other, muscle and tissue are lifted from the desired medical site, allowing vision and access to the desired medical site. The desired medical site may be any acceptable medical site, such as a vertebra (440) or other location to which a surgeon desires to have clear and clean access. Looking from the proximal end (140), a view of the desired medical site is unobstructed by muscle and tissue that previously covered the site.

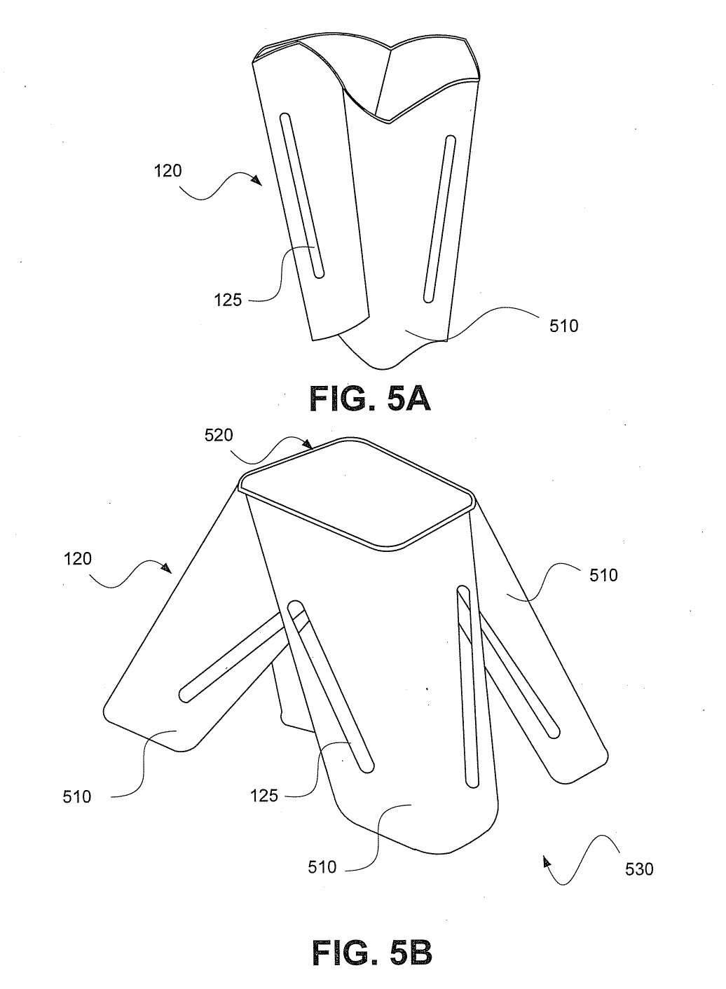

FIG. 5A illustrates an exemplary retractor assembly (120) that may be used with the present less invasive access port device (100; FIG. 1), according to one exemplary embodiment. As illustrated in FIG. 5A, the retractor assembly (120) is shown with retractor members (510); as illustrated the exemplary retractor assembly is configured with four retractor members connected at the proximal end (520, FIG. 5B). The retractor members each have two slots (125, FIG. 5B), and as can be clearly seen, the slots align when the retractor members (510) are closed as in FIG. 5A. With the retractor members closed as in FIG. 5A a pin inserted in the distal end of the slot (125) will prevent the retractor members (510) from expanding. Upon insertion of the retractor assembly into a desired medical site, the pin is slid from the distal portion of the slot (125) towards the proximal end of the slot (125); as the pin is slid up the retractor members (510) expand and muscle and tissue is pushed out of the work site. Thus, a working area is cleared and a surgeon is able to clearly see the work site.

As described in previous paragraphs as well as will be described in subsequent paragraphs, a pin is used within the slots (125) of retractor members (510, FIGS. 5A and 5B); the pin is not shown and may consist of any member that facilitates in securing the retractor members anywhere from a closed and contracted state (FIG. 5A) to a open and expanded state (FIG. 5B) or to a point in the middle, a partially expanded state. Additionally the pin may include an attachment allowing easier access to the pin, such as a member extending upwards within or out of the retractor assembly allowing a surgeon to manipulate the location of the pin within the slot (125).

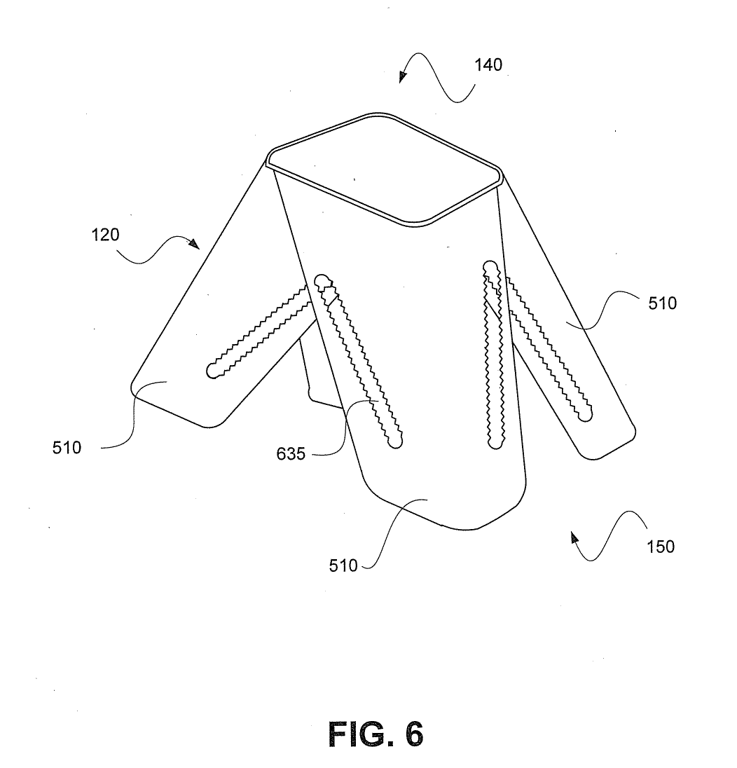

FIG. 6 illustrates and alternative embodiment of the slots (125) of previous figures. In this embodiment the slots (635) of the retractor members (510) are configured with teeth. The teeth or other frictional members, allow a pin to be ratcheted within the slot (135) to a desired location. A pin in the lowest possible location would place the retractor members (510) in a closed position, similar to that as illustrated in FIG. 5A. A pin moved or ratcheted to the upper most position (towards the proximal end (140)) would place the retractor members (510) in a completely open and expanded position. In this embodiment, where the slots (635) are configured with teeth, the pin may also be placed anywhere in the middle of the slot and secured there by the teeth; in a position other than the upper most or lower most position, the retractor members would be placed in a partially expanded state. Thus allowing medical personal access to a desired medical site while minimally impacting surrounding tissue. Various modifications may be made to the slot and pin configuration described; however, it is desired that the pin and the slot are configured in such a way so as to allow the pin to be placed at various positions between the upper most and lower most position, this may include pins of shapes configured to facilitate in securing the pin in a location within the slot.

The retractor assembly (120) may also be configured having an optional soft tissue barrier. According to one exemplary embodiment, a flexible material may be added to the retractor members (510) such that when the retractor members are deployed, the open space between the retractor members (510) are occupied by the soft tissue barrier. The soft tissue barrier may be added between the retractor members (510), according to one exemplary embodiment, to ensure that soft tissue does not herniate into the working channel when the retractor blades (510) are deployed.

While the retractor members (510) of the exemplary retractor assembly (120) have been described above and illustrated in the Figures as having a particular shape, the retractor blades (510) of the retractor assembly (120) may assume any number of shapes, and may be made of any number of materials to satisfy a desired surgical purpose.

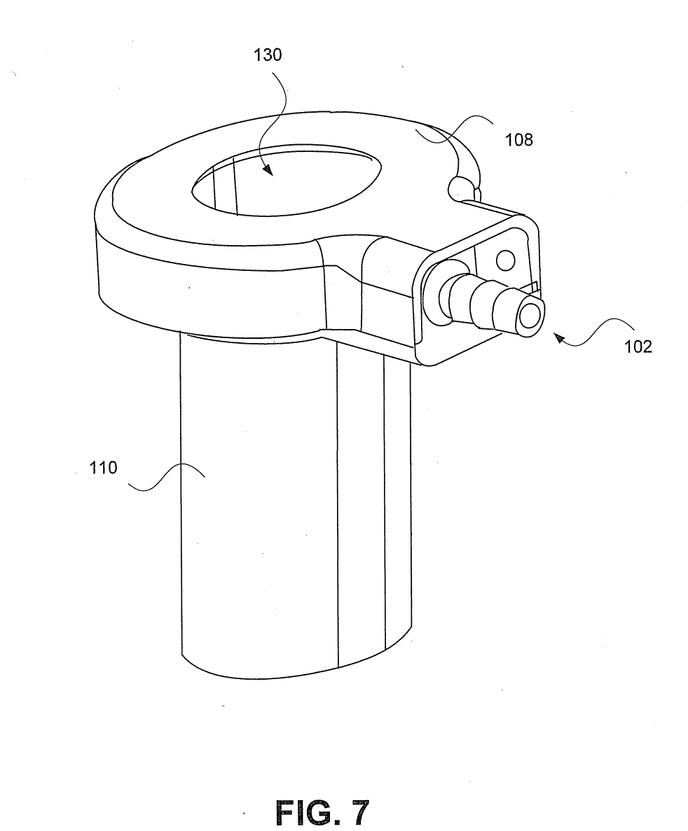

Continuing with the components of the exemplary less invasive access port device (100; FIG. 1), FIG. 7 is an isometric view illustrating an exemplary cannula assembly, a cannula sleeve (110) and a coupled housing (108), prior to engagement with the retractor assembly (120; FIG. 1). As shown, the exemplary cannula sleeve defines an access port (130), includes a housing (108) on a proximal end of the cannula sleeve (110). According to the exemplary embodiment illustrated in FIG. 7, the housing (108) includes integrated interfaces (102) for fiber optic lights, irrigation, and suction. According to one exemplary embodiment illustrated in FIG. 7, the access port (130) defined by the body of the cannula sleeve (110) is sufficiently large and of an appropriate geometry to allow for the passage of a number of operating tools to access an identified surgical location. Additionally, the access port (130) may also provide an optical inspection portal, allowing a surgeon to visually inspect the identified surgical location without the use of optical cameras and the like.

The cannula sleeve (110), according to one exemplary embodiment, is flexible allowing the sleeve to be positioned at a desired angle relative to the retractor assembly (120). After the cannula sleeve (110) and coupled housing (108) is attached to the retractor assembly (120) the cannula sleeve may be positioned flexibly to any desired angle allowing access to the entire work area provided by the expanded retractor members (510, FIG. 5B).

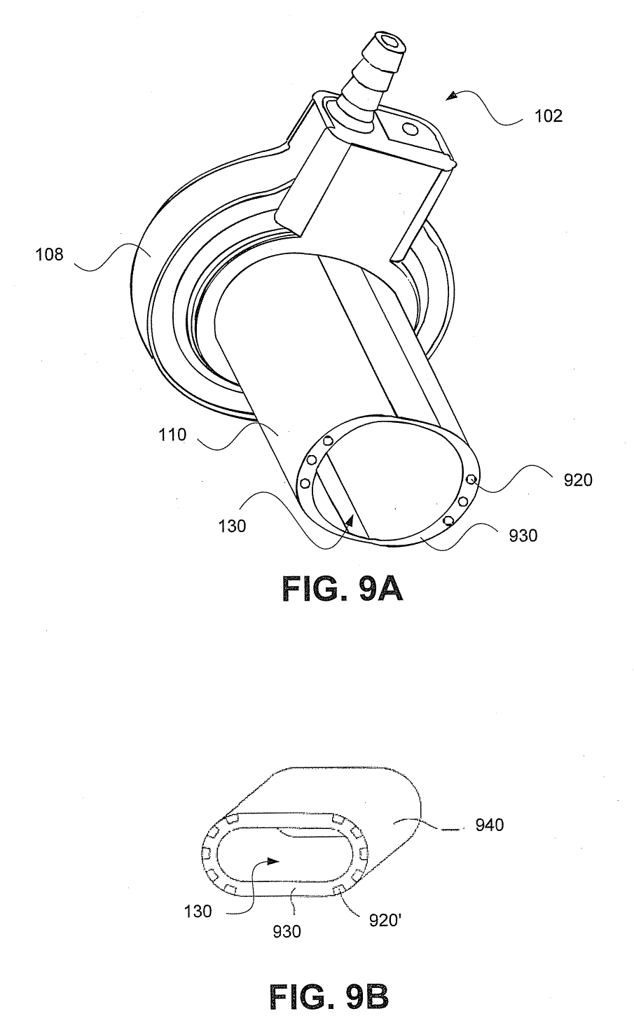

According to one embodiment with a flexible cannula sleeve (110) the cannula sleeve is attached to the retractor assembly (120) in any way that is convenient. According to one embodiment, the cannula sleeve has an outer perimeter allowing the cannula sleeve (110) to enter partially into the retractor assembly (120) and therein be secured by locking mechanisms such as protrusions and corresponding grooves or orifices. According to another embodiment, the cannula sleeve (110) fits around the outer portion of the proximal end (140) of the retractor assembly (120) and there is secured by protrusions and corresponding grooves or orifices. According to yet another embodiment the cannula sleeve (110) neither slides within or around the retractor assembly (120), but rather mates the bottom rim of the cannula sleeve (930, FIG. 9A) with the upper rim of the retractor assembly (120) with corresponding protrusions and grooves.

An alternative embodiment, shown and described in detail below, provides a flexible member that interconnects the cannula sleeve (110) and the retractor assembly (120). This flexible member allows the cannula sleeve to be flexibly pivoted to an angle relative to the retractor assembly (120) rather than having a flexible cannula sleeve (110).

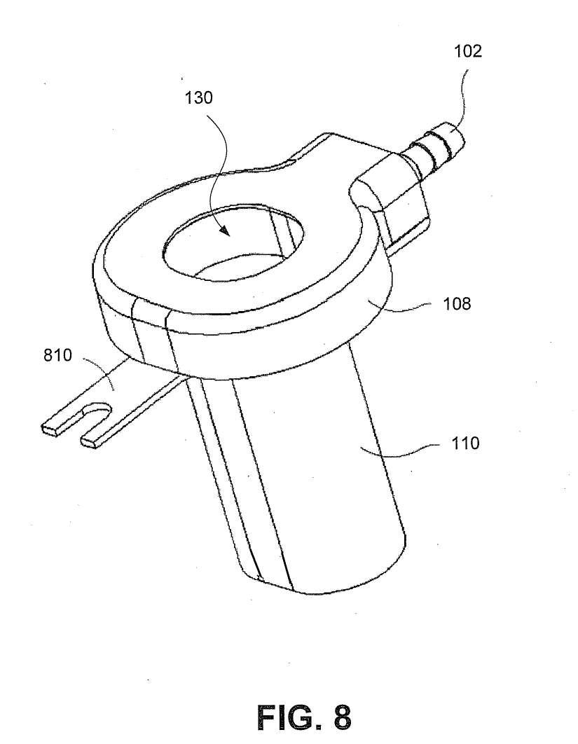

FIG. 8 shows an alternative embodiment of a cannula sleeve (110) coupled to a housing (108) with a leyla arm attachment (810) coupled thereto. The attachment (810) serves as a mount for attachment of the housing (108) to a positioning arm during an operation. In alternative embodiments, mounts of various size and configuration as are known in the art and could be added to the housing.

FIG. 9A is a bottom isometric view of the cannula sleeve (110) and coupled housing (108), according to one exemplary embodiment. As illustrated in FIG. 9A, a number of channels (920) are contained in the cannula wall (930) connecting the work site with the housing (108) at a proximal end of the cannula sleeve (110). According to one exemplary embodiment, aspiration and irrigation of the work site is accomplished through the channels (920) or passages in the distal face of the cannula sleeve (110). The integrated interfaces (102) are contained on the housing (108) and connect to the channels (920) to support the aspiration and irrigation at the work site. Additionally, according to one exemplary embodiment, light can be supplied to the cannula sleeve (110), and consequently the work site, through a fiber-optic cable, similar to that used with surgical headlamps. According to one exemplary embodiment, the fiber optic cables are truncated at the distal face of the cannula sleeve (110). According to this exemplary embodiment, light from a fiber optic cable will pass down the wall of the cannula sleeve (110), as it would a fiber-optic cable, to illuminate the work site.

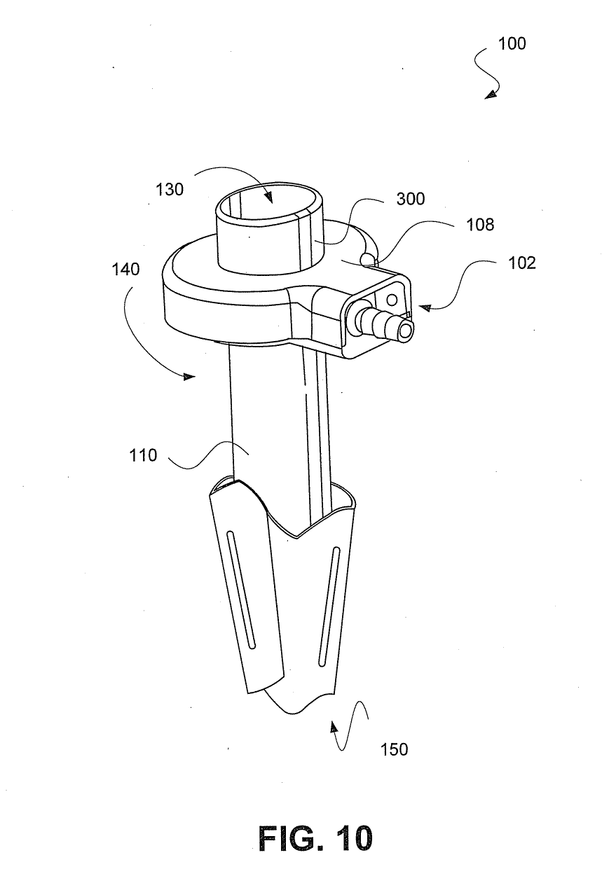

While the channels (920) may be drilled or otherwise formed in the cannula sleeve wall (930), FIG. 9B illustrates an alternative embodiment of the cannula sleeve wall (930). According to the exemplary embodiment illustrated in FIG. 9B, the cannula sleeve (940) includes a cannula wall (930) defining an access port (130). The outer surface of the cannula wall (930) includes a plurality of ridges or fins defining slots (920′) in the exterior cannula wall (930). Further, a cannula sleeve (940) or sheath is formed over the outside of the cannula wall (930) to seal the fins or slots (920′) contained on an outside surface of the cannula wall (930). The slots (920′) contained on the outside surface of the cannula wall (930) may be ridges, grooves, channels, fins or the like. The slots (920′) provide a passage for aspiration, the placement of fiber optic filaments as a light source, video feed, or the like. In accordance with aspects of the present exemplary embodiment, the cannula sleeve (110) may be made out of a light transmitting material to channel light into the working space through the walls of the cannula. Assembly and deployment of the exemplary less invasive access port device (100; FIG. 1) will now be described with reference to FIGS. 10 through 16.

As mentioned previously, a k-wire may be inserted, with the aid of a fluoroscope, into a desired working space. Any number of pedicle screws may then be percutaneously inserted into a desired bone mass. A trocar (300) may then be placed over the k-wire to dilate the soft tissues and provide access to a desired working site. With the trocar appropriately placed, a retractor assembly (120) can be introduced over the trocar (300) and down to the working site (not shown). As illustrated in FIG. 10, the retractor assembly (120) in its un-deployed configuration retains the retractor members (510) adjacent to one another, forming a channel. The trocar (200) can be received within the distal opening of the channel and the retractor assembly (120) may then be slid down the trocar (300) in its undepolyed state until the distal portion (150) of the retractor is in a desired working space.

With the retractor assembly (120) correctly positioned in the desired working space, the cannula sleeve (110) may also be introduced over the trocar (300) until it engages the retractor assembly. FIG. 10 illustrates an exemplary cannula sleeve (110) and coupled housing (108) introduced over the trocar (300). As illustrated, the retractor assembly (120) has not been deployed, and thus pins within the slots (125 or 135) remain in a position securing the retractor members (510) in a closed contracted state. As shown in FIG. 10, the trocar (300) is received through the access port (130) of the cannula sleeve (110).

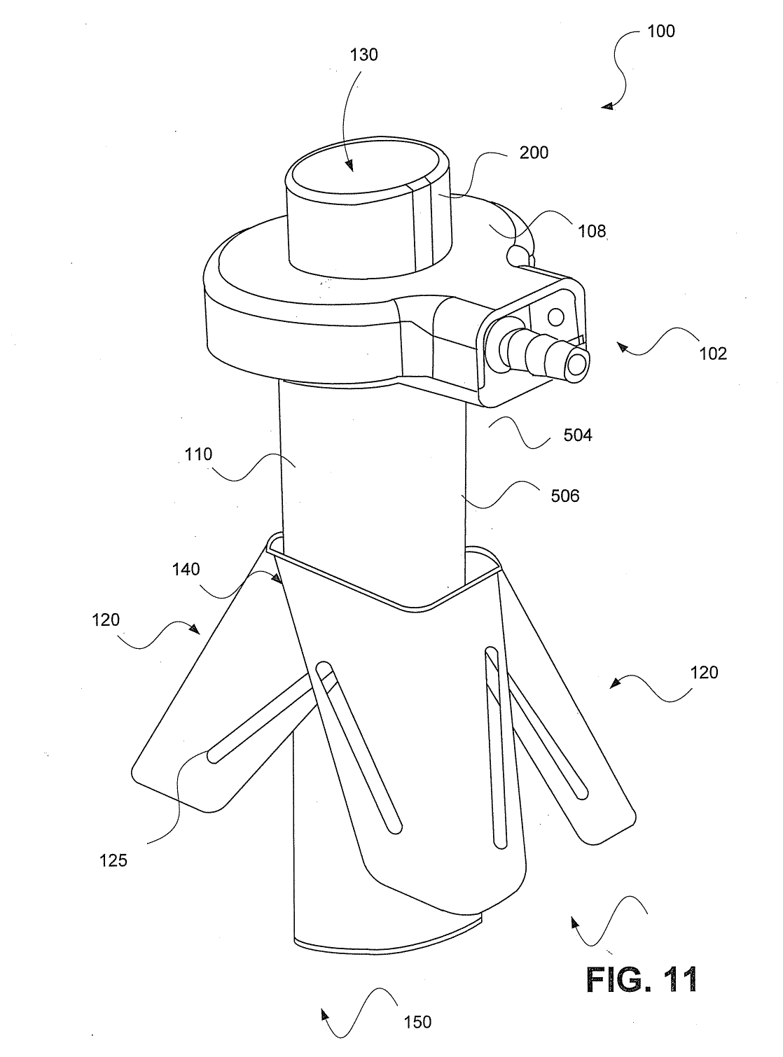

FIG. 11 shows the less invasive access port device (100) in a deployed position prior to removal of the trocar (300) from the assembly. As shown the pins placed in the highest or most proximal position within the slots (125 or 135) allowing the retractor members (510) to expand near the distal end (150); thus, the retractor opens to further dilate the soft tissues at the working site. With the retractor assembly (120) in a deployed position, the trocar (300) may be removed and the working site may be manipulated.

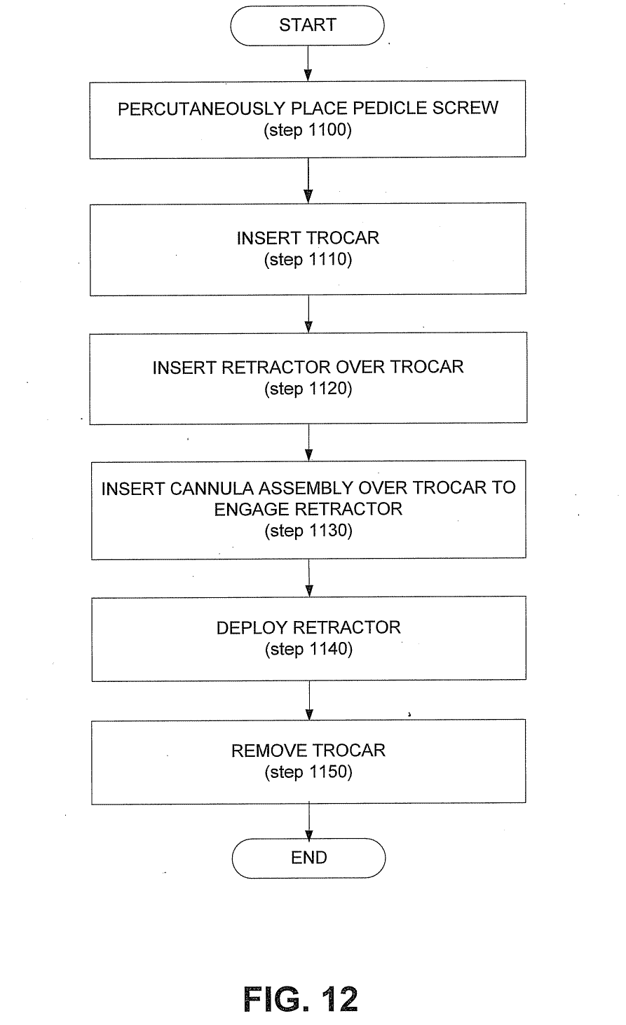

According to one exemplary embodiment, the retractor assembly (120) can be diametrically expanded after it is deployed. This will increase the working area/channel within the retractor. Any appropriate expanding instrument could be used. Further details of the implementation and operation of the less invasive access port device (100) will be provided below with reference to FIGS. 12 through 16.

### Exemplary Implementation and Operation

FIG. 12 illustrates an exemplary method for using the present exemplary less invasive access port device (100) to access a desired work site on a patient's spine. As illustrated in FIG. 12, the exemplary method begins by first percutaneously placing one or more pedicle screws in vertebra (step 1100). With the pedicle screws in place, a trocar or other dilating device may be inserted at the location of the pedicle screw (step 1110). With the trocar in place, a retractor assembly is slideably inserted over the trocar (step 1120), followed by the insertion of a cannula assembly over the trocar to engage the retractor assembly (step 1130). With the less invasive access port device (100; FIG. 1) assembled, the retractor may then be deployed (step 1140) followed by the removal of the trocar (step 1150).

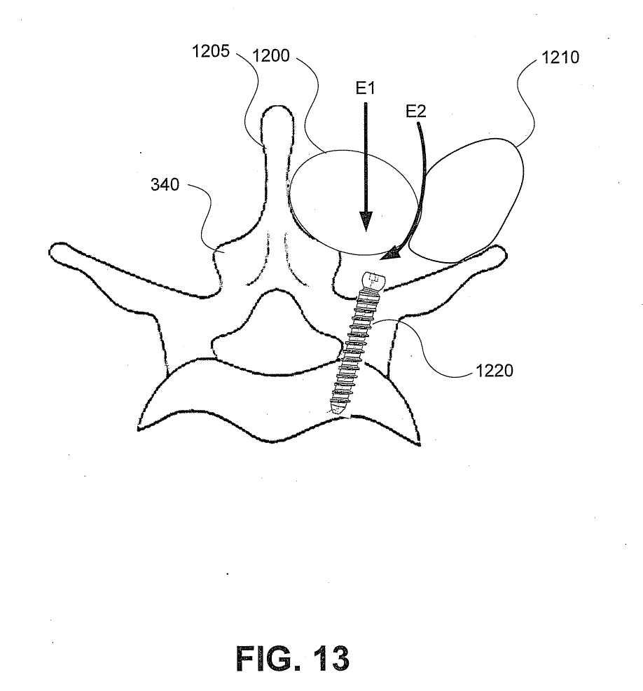

As mentioned above, the present exemplary method includes inserting one or more pedicle screws in a patient's vertebra (step 1100) prior to the insertion of a trocar or cannula sleeve. According to one exemplary embodiment, the percutaneous insertion of one or more pedicle screws (step 1100), the insertion of the trocar (step 1120), and the insertion of the retractor over the trocar (step 1130) is performed in the plane lateral to the multifidus. As illustrated in FIG. 13, the lumbar vertebra (340) have a number of muscle groups that run on top of the vertebra. As shown in FIG. 13, the multifidus (1200) is located adjacent to the spinous process (1205). The longissimus muscle group (1210) is positioned lateral to the multifidus (1200). Current MIS approaches insert pedicle screws and their associated hardware through an entry path that traversed the multifidus muscle group (1200), as illustrated by E1. This technique unnecessarily damages soft tissue, resulting in pain and increased rehabilitation for the patient. According to the present exemplary embodiment, the entry path illustrated by E2 is used for the insertion of the pedicle screw, a trocar, or a cannula.

Specifically, insertion of one or more pedicle screws in a patient's vertebra (step 1100) includes performing a blunt dissection in the plane lateral to the multifidus (1200) approaching the area of the transverse process where it reaches the lateral aspect of the facet joint. Then, under fluoroscopic guidance, a screwdriver, screw/sleeve assembly with or without a sleeve (not shown) can be used to place the pedicle screw (1220) in the vertebra (340).

With the pedicle screw(s) (1220) in place, a trocar or other sleeve may be inserted, in the plane lateral to the multifidus, to the location of the pedicle screw(s) (step 1110). Insertion of the trocar dilates the soft tissue, allowing the formation of a working space. With the trocar appropriately placed, the retractor assembly (120; FIG. 1) is placed over the trocar and slideably inserted into the working space (step 1120). As mentioned previously, when the retractor assembly (120; FIG. 1) is positioned within the working space, the pins within the slots are easily accessible to a surgeon, allowing the surgeon to expand the retractor members when desired. This allows the two-part retractor to be easily locked in a deployed position.

With the retractor properly placed, the cannula assembly may be placed over the trocar and engaged with the retractor (step 1130) followed by deployment of the retractor (step 1140). According to one exemplary embodiment, the deployment of the retractor and engagement of the cannula sleeve with the retractor may be performed in any order. According to one exemplary embodiment, when the retractor assembly is deployed (step 1140), the muscles surrounding the working space are retracted. Prior to deploying the retractor, a series of Cobb elevators and other instruments could be used to subperiosteally dissect the muscle off the facet joints and lamina and spinous processes creating a working space for the retractor to be deployed in.

When the retractor is deployed in the working space, the trocar and any other sleeves may be removed from the access port of the less invasive access port device (step 1150). Once removed, the working space may be accessed for performing decompression, discectomy, interbody fusion, partial facetectomy, neural foraminotomy, facet fusion, posterolateral fusion, spinous process removal, placement of interspinous process distractors, or facet replacement, pedicle replacement, posterior lumbar disc replacement, or any one of a number of other procedures.

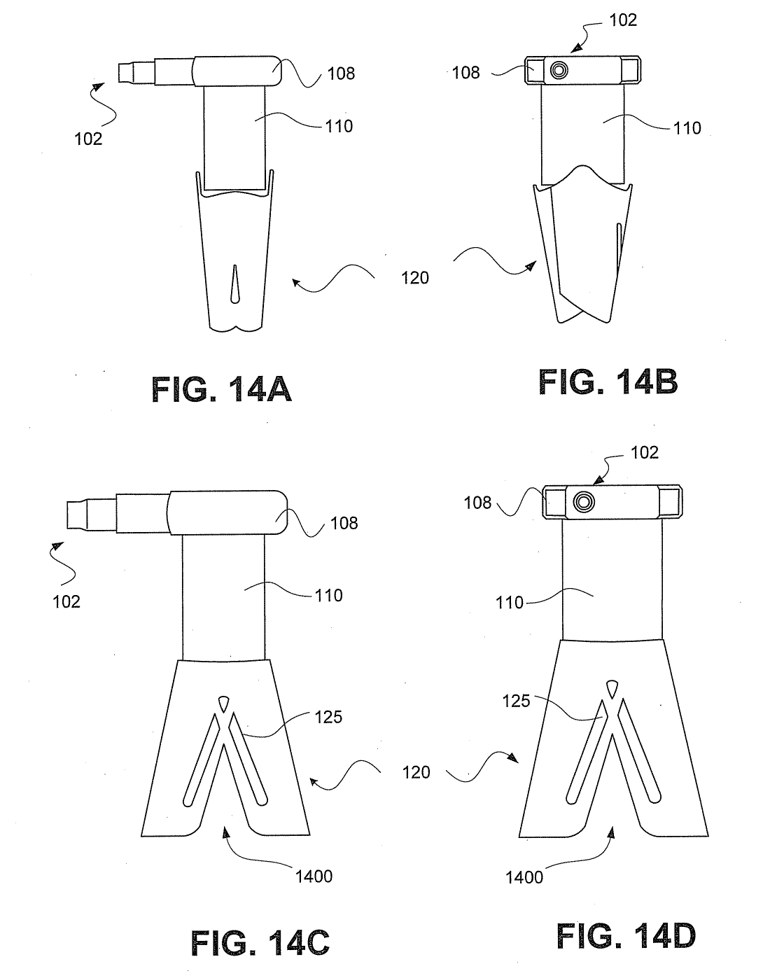

FIGS. 14A and 14B show the complete less invasive access port assembled in an undeployed state, retractor assembly (120) contracted. As previously mentioned each of the retractor members (510, FIGS. 5A and 5B) is secured in a position either completely expanded, completely contracted, or partially expanded. By utilizing pins that may be ratcheted within a slot (635, FIG. 6), the retractor assembly provides controlled, variable dilation, both medial-lateral and superior-inferior. It is possible to dilate in a medial-lateral direction fully by expanding two retractor members (510) fully, while dilating in a superior-inferior direction only partially expanding two retractor members.

FIGS. 14A and 14B show a two side views of a less invasive access port assembled in a deployed state, retractor assembly (120) fully expanded. It is of note that when fully expanded, the retractor members provide gaps (1400) that allow access to extra-dilated space for contra-lateral decompression, passing of transverse connector, etc. If necessity warrants however, a soft tissue barrier may be placed within the gaps (1400) preventing tissue from herniating into the access area.

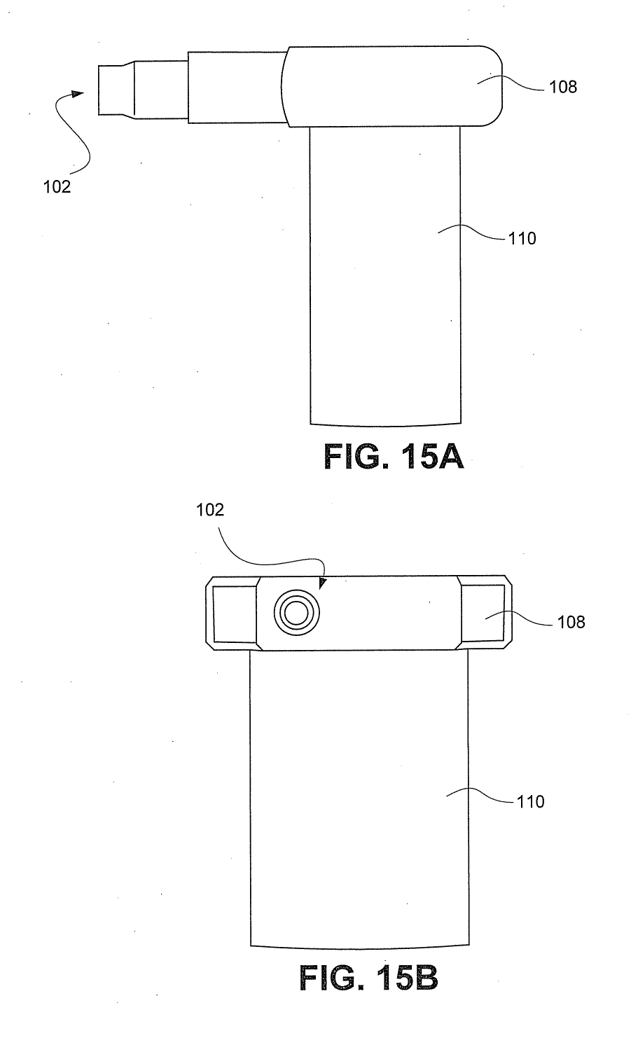

FIGS. 15A and 15B illustrate again the cannula sleeve (110) and coupled housing (108) from two side views. As the cannula sleeve may provide illumination, suction, aspiration, irrigation, and or fiber optic channels for light or video, it may be desired to use the cannula sleeve (110) and housing (108) exclusive of the retractor assembly (120). It may be desired to use the cannula sleeve (110) as a portal to isolate a working site with the walls of the cannula sleeve and provide light, irrigation, or aspiration to the site, while not necessarily utilizing a retractor assembly (120) to secure muscle or other tissue from the site; this may be accomplished by utilizing the cannula sleeve (110) in conjunction with the housing (108) and integrated interfaces (102).

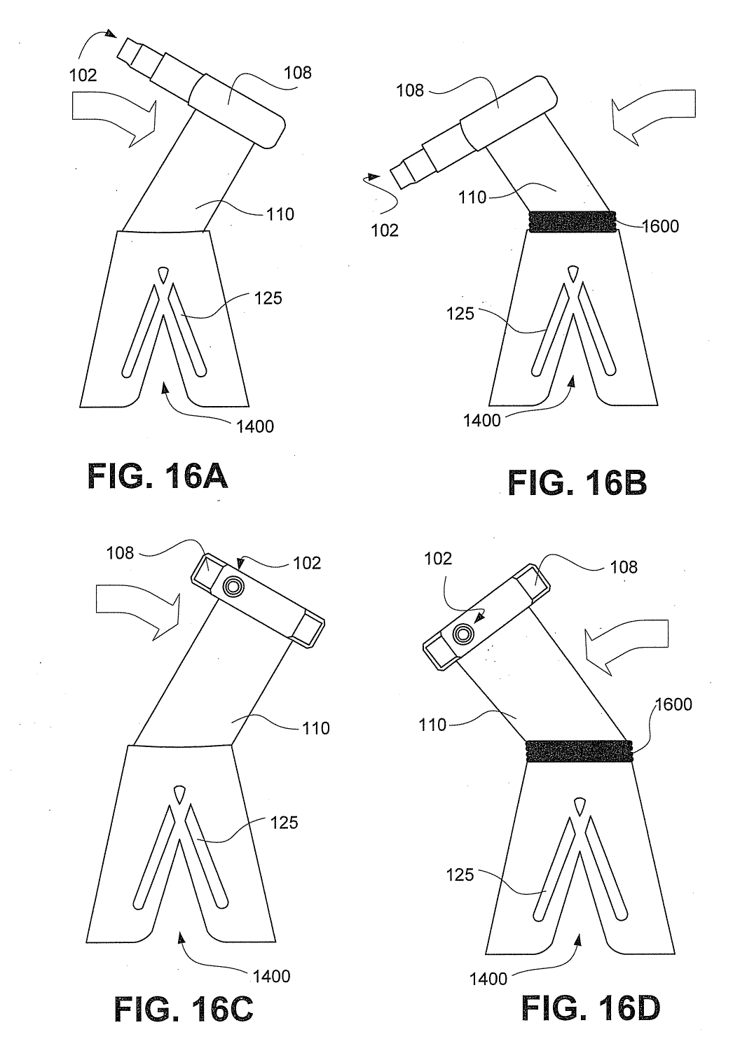

Performance of the various procedures via the access port (130; FIG. 1) is facilitated by the rotational freedom provided by the present less invasive access port device (100; FIG. 1). FIGS. 16A-16D illustrate the angulation of the cannula sleeve (110) within the retractor assembly (120). Specifically, according to one exemplary embodiment, the motion of the cannula sleeve (110) within the retractor assembly (120) may be facilitated by a number of elements. According to a first exemplary embodiment, the cannula sleeve (110) mates with the retractor assembly in one of the manners described above.

Alternatively, the cannula sleeve (110), is coupled to the retractor assembly (120) by a flexible member configured to allow the cannula sleeve (110) to be positioned in any angle desired relative to the retractor assembly (120). Specifically, according to one exemplary embodiment, the flexible member may be configured to be flexed and then return to a specific angle once released, or alternatively may be constructed of a material allowing it to be flexibly positioned to a specific angle and when released retain that angle until further acted upon. As shown in FIGS. 16A and 16C, the flexible member (not shown) may couple the cannula sleeve (110) and the retractor assembly (120) within the base of the retractor assembly. Coupling the flexible member within the retractor assembly (120) allows the cannula sleeve (110) to be free to pivot as shown by the arrows in FIGS. 16A and 16B.

Alternatively, the cannula sleeve (110) may be coupled directly to the retractor assembly (120) by a flexible connecter (1600) configured to secure the proximal portion of the retractor assembly (120) to the distal end of the cannula sleeve (110), as is illustrated in FIGS. 16B and 16D. According to one exemplary embodiment, the flexible connector (1600) may be made of any number of flexible materials including, but in no way limited to, rubber or plastics such as polyolefin or PVC heat shrink tubing. According to this exemplary embodiment, the rubber connection member (1600) may attach in any number of ways to both the retractor assembly (120) and the cannula sleeve (110). According to one exemplary embodiment, the rubber connection member (1600) may be attached via adhesives, fasteners, friction fits, and any other appropriate connection system or method. It is conceivable, according to one exemplary embodiment, that the rubber member (1600) is permanently attached to either the cannula sleeve (110) or the retractor assembly (120) and is therefore detachably connected to the other portion of the access port. It is also within the scope of the present exemplary system and method to provide a rigid cannula sleeve (110) attached directly to the retractor assembly (120) providing pivotable motion; or, using the same embodiment, place a rubber member (1600) in between the cannula sleeve (110) and the retractor assembly (120). The present exemplary cannula sleeve (110) including the rubber or flexible member (1600) allows the cannula sleeve to flex in the superior/inferior directions as well as the mediolateral direction relative to the retractor assembly (120). Particularly, the use of a flexible sleeve such as a rubber connector (1600) adjoining proximal tube to distal speculum provides for any desired relative motion; the proximal tube can pivot omnidirectionally relative to the distal speculum.

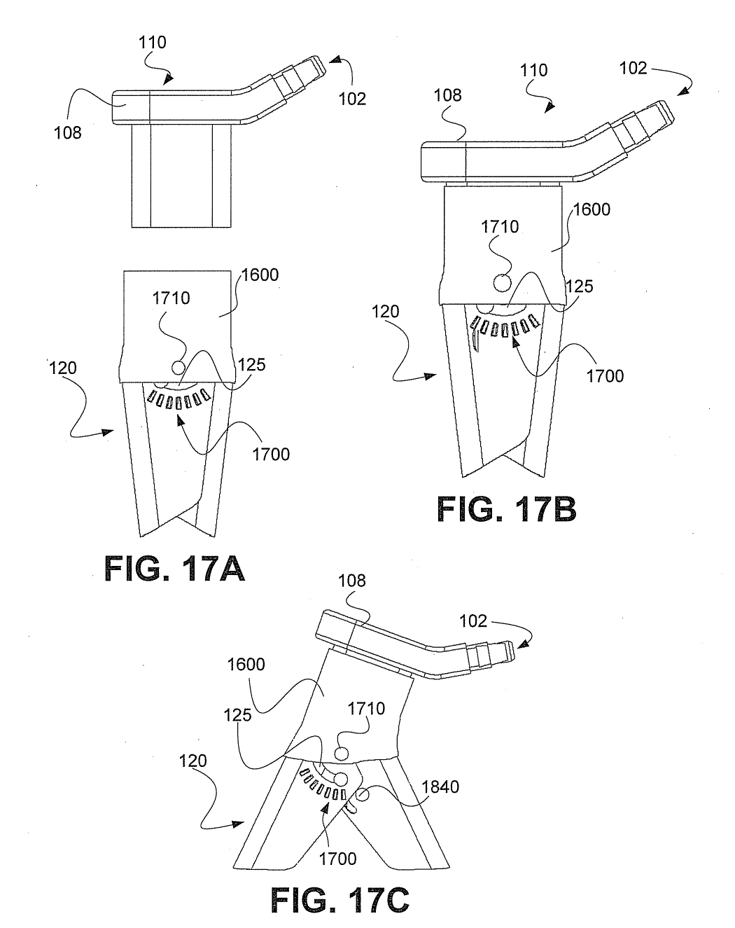

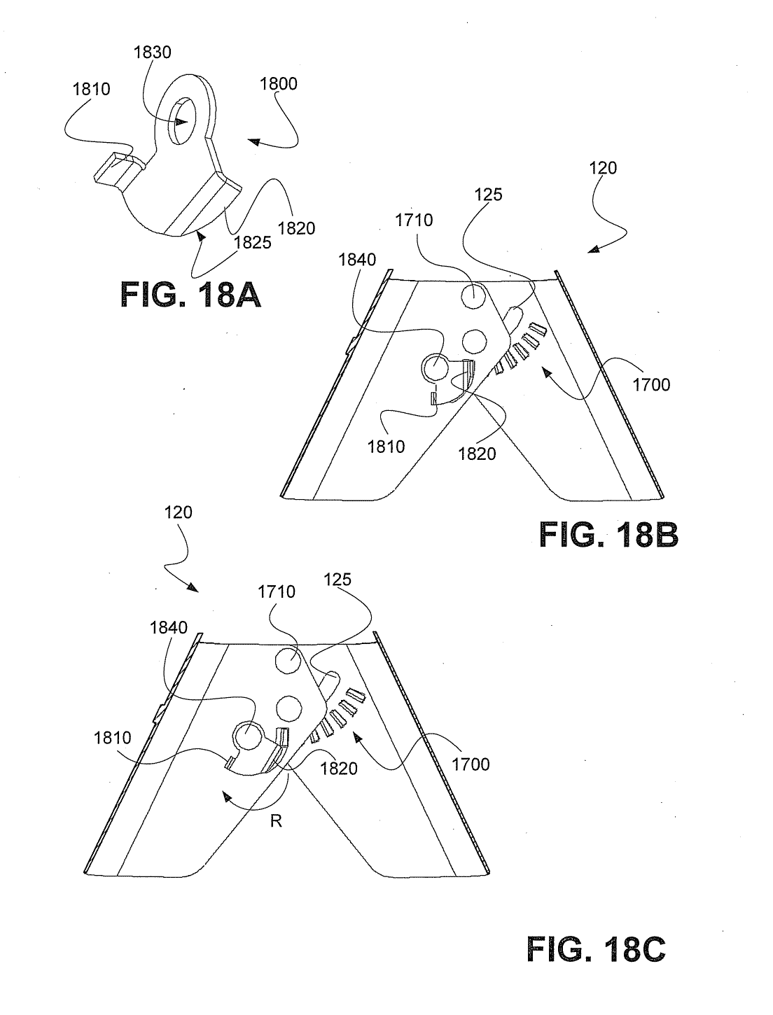

FIGS. 17A through 18C further illustrate a less invasive access device (100; FIG. 1A) according to an alternative embodiment. As illustrated in FIGS. 17A through 17C, the exemplary less invasive access device includes a cannula sleeve (110) having a housing (108) and a number of integrated interfaces (102) as described above. Additionally, similar to the exemplary embodiments described above, the exemplary less invasive access device includes a retractor assembly (120) including a flexible member (1600) coupled to the proximal end of the retractor assembly. Furthermore, as shown, the exemplary less invasive access device includes a plurality of retractor blades joined at a pivot pin (1710) and include a pin and slot (125) connection for facilitating the selective spreading of the retractor blades. However, in contrast to the previously disclosed systems and methods, the exemplary embodiment illustrated in FIGS. 17A through 18C include a plurality of ratchet slots (1700) formed on the sides of at least one retractor blade. As illustrated in FIGS. 17A through 17C, the exemplary ratchet slots (1700) are formed in an arcuate pattern such that a point on the underlying retractor blade is aligned with at least one ratchet slot as the retractor blades are transitioned from a closed position to a spread or open position.

FIGS. 18A through 18C further illustrate the exemplary components of the present exemplary less invasive access device. FIG. 18A illustrates a ratchet latch (1800) that is fastened to the underside of the internal retractor blade adjacent to the ratchet slots (1700), according to one exemplary embodiment. As illustrated in FIG. 18A, the exemplary ratchet latch (1800) defines a fastener orifice (1830) defined therein. Additionally, as illustrated, a manipulation tab (1810) is formed in a first direction from the main body and a ratchet tooth (1820) is formed in a second direction, opposite the direction of the manipulation tab. According to one exemplary embodiment, the ratchet tab is formed with a rounded face terminating in a substantially 90 degree corner or tooth. FIG. 18B further illustrates the assembly of the exemplary ratchet latch (1800) on the exemplary less invasive access device. As illustrated in the exemplary cross-sectional view of FIG. 18B, the ratchet latch (1800) is coupled to the internal surface of the retractor blade by a latch fastener (1840) that passes through the fastener orifice (1830). Consequently, the ratchet latch (1800) is rotatably coupled to the internal surface of the retractor blade. Additionally, a single ratchet slot (1700) is formed on the internal surface of the retractor blade such that at least a portion of the ratchet tooth (1820) passes there through and into the ratchet slots (1700) on the opposing retractor blade. According to one exemplary embodiment, the passing of the ratchet tooth through and into the ratchet slots (1700) creates an interference fit between the two retractor blades, thereby positionally securing the relative position of the two retractor blades. Due to the relatively arcuate facial surface (1825) of the ratchet tooth, separation of the retractor blades is permitted once an initial resistance is overcome. However, the 90 degree corner of the tooth strictly resists the collapse of the retractor blades. Consequently, once a desired position of the retractor blades is established, the present exemplary system maintains the desired position.

FIG. 18C illustrates the disengagement of the exemplary ratchet latch (1800) when collapse of the retractor blades is desired. As illustrated in FIG. 18C, a force may be exerted on the manipulation tab (1810) causing the ratchet latch to rotate (R). As the ratchet latch is rotated (R), the ratchet tooth is withdrawn from the ratchet slots (1700) removing the interference that prevents closure of the retractor blades. Consequently, the retractor blades can be closed and the assembly removed.

Further advantages of the present exemplary system include the variety of materials, including composites, plastics and radio-opaque materials, that the cannula and retractor can be made from. Existing MIS access ports are made of metal, which has several shortcomings: metal conducts electricity which can cause arcing from an electrocautery device and thus unwanted stimulation of the nerves; metals are reflective and produce an environment that is difficult to clearly view the surgical site; metals are radio-opaque and make intra-operative x-ray difficult. Alternative materials that are partially radio-opaque would provide for optimal intra-operative x-ray. The geometry and structural integrity of the prior art does not allow for the use of alternative materials.

In conclusion, the present exemplary systems and methods allow for a surgeon to manipulate the viewing angle of the less invasive access port into the working site in a transverse plane. Manipulation of a port medially and laterally facilitates: decompression of the neural elements; simple access to the contralateral side of the spine, eliminating the need to place a tube through the skin on that side; access to the transverse process on the ipsalateral side for a posterolateral fusion, and generally simplifies a surgical procedure by increasing the surgeon's viewing of the surgical site. Further, the present exemplary systems and methods allow for the retraction of muscles rather than the distal lifting of muscles during procedures. Additionally, the present exemplary system positions the arm securing mechanism outside of the wound where it may be readily accessed by the surgeon.

Moreover, the present system and method do not require the additional use of a light source, a suction device, and an irrigation device because these items are integral to the construction of the less invasive access port device. Existing MIS access ports require the additional use of a light source, a suction device, and an irrigation device, all of which decrease the space left for surgical instruments and for viewing of the surgical site.

The preceding description has been presented only to illustrate and describe the present method and system. It is not intended to be exhaustive or to limit the present system and method to any precise form disclosed. Many modifications and variations are possible in light of the above teaching.

The foregoing embodiments were chosen and described in order to illustrate principles of the system and method as well as some practical applications. The preceding description enables others skilled in the art to utilize the method and system in various embodiments and with various modifications as are suited to the particular use contemplated. It is intended that the scope of the present exemplary system and method be defined by the following claims.

What is claimed is:

1. A less invasive access port, comprising:
a retractor assembly including a plurality of retractor blades, wherein at least one retractor blade includes a plurality of positioning slots;
a cannula having walls, said cannula configured to be coupled to said retractor assembly.

2. The less invasive access port of claim 1, wherein said plurality of positioning slots of said retractor blades are configured to receive a pin;
wherein said pin is configured to be variably positioned within said plurality of positioning slots to secure said retractor blades in a desired position.

3. The less invasive access port of claim 2 wherein said plurality of positioning slots in said retractor blades further comprise a plurality of teeth configured to allow said pin to be selectively placed in a plurality of positions.

4. The less invasive access port of claim 1 further comprising a channel defined in a wall of said cannula, wherein said channel is configured to fluidly connect a top portion of said cannula to a bottom portion of said cannula.

5. The less invasive access port of claim 4 wherein said cannula walls are configured to transmit light.

6. The less invasive access port of claim 4, further comprising a housing fluidly connected to said channel, wherein said housing includes at least one integrated interface port.

7. The less invasive access port of claim 1, wherein said cannula comprises a flexible material;
said cannula being configured to be positioned at a desired angle relative to said retractor assembly.

8. The less invasive access port of claim 1, wherein said cannula is coupled to a flexible member, wherein said flexible member is further coupled to said retractor assembly; said flexible member being configured to provide a plurality of degrees of freedom of said cannula relative to said retractor assembly.

9. A retractor comprising:
a first and a second retractor blade;
at least one positioning slot on each of said first and second retractor blade;
at least one pin configured to be inserted into said slots in a first and second position, wherein said first position secures said retractor blades in a contracted closed state and said second position secures said retractor blades in a deployed expanded state.

10. The retractor of claim 9, wherein said slot further comprises a plurality of teeth configured to selectively position said pin in one of a plurality of positions in said slot.

11. The retractor of claim 9, wherein said retractor comprises:
a first, a second, a third and a fourth retractor blade positioned adjacently to form a closed loop;
wherein each retractor blade has a first and a second slot, said first slot aligning with a slot on the retractor blade to the left and a second slot aligning to a slot on retractor blade to the right;
wherein each of said first, second, third, and fourth retractor blade is coupled to two adjacent retractor blades through a slot and a pin.

12. The retractor of claim 11, wherein said slots on said retractor blades further comprises a plurality of teeth configured to selectively position said pin in one of a plurality of positions in said slot.

13. The retractor of claim 9, further comprising a flexible orientation member coupling said retractor to a cannula;
wherein said flexible orientation member is configured to allow said cannula to be securely attached to said retractor while allowing said cannula to be pivoted in any direction relative to said retractor

14. A less invasive access port, comprising:
a retractor assembly including at least a first and a second retractor blade;
a flexible coupling member; and
a cannula configured to be coupled to said retractor assembly via said flexible coupling member;
wherein said cannula is configured to pivot to any desired angle relative to said retractor assembly.

15. The less invasive access port of claim 14, wherein said flexible coupling member comprises at least a portion of said cannula formed of a flexible material.

16. The less invasive access port of claim 14, wherein said retractor assembly further comprises:
a first, a second, a third and a fourth retractor blade positioned adjacently to form a closed loop;
wherein each retractor blade has a first and a second slot, said first slot aligning with a slot on the retractor blade to the left and a second slot aligning to a slot on retractor blade to the right;
wherein each of said first, second, third, and fourth retractor blade is coupled to two adjacent retractor blades through a slot and a pin.

17. An access port, comprising:
at least one retraction member, said at least one retraction member being configured to displace tissue;
an entry member configured to provide an initial opening to said access port; and
a flexible joining member flexibly coupling said at least one retraction member to said entry member, said flexible joining member facilitating superior and inferior movement as well as mediolateral movement of said entry member relative to said at least one retraction member.

18. The access port of claim 17, wherein said at least one retraction member is configured to lock in at least three fixed positions.

19. The access port of claim 18, further comprising:
a plurality of retraction members; and
a locking latch disposed between said retraction members;
wherein said latch includes a planar body, a ratcheting member protruding substantially perpendicularly from a first side of said planar body and a tab member protruding from a second side substantially perpendicular to said body, wherein said ratcheting member is configured to engage at least one engagement feature on said plurality of retraction members to fix said position; and
wherein said tab is configured to facilitate release of said ratcheting member from said at least one engagement feature.

20. The access port of claim 19, wherein said at least one engagement feature on said plurality of retraction members comprises a plurality of ratchet slots defined by said plurality of retraction member, said ratchet slots formed in an arcuate configuration.

21. The access port of claim 18, wherein said ratcheting member includes a generally arcuate surface terminating in a 90 degree corner.
